(19) United States
(12) Patent Application Publication
(10) Pub. No.: US 2015/0005886 A1
(43) Pub. Date: Jan. 1, 2015

(54) Prosthetic Joints

(71) Applicant: John Michael Pinneo, Portola Valley, CA (US)

(72) Inventor: John Michael Pinneo, Portola Valley, CA (US)

(21) Appl. No.: 13/933,061

(22) Filed: Jul. 1, 2013

(51) Int. Cl. A61F 2/30

(52) U.S. Cl. CPC A61F 2/30767; A61F 2002/30079
USPC 623/18.12

(57) ABSTRACT

Methods are described for improving the performance of implanted prosthetic joints through the use of magnetic technology. Arrays of magnets are employed to modify static and/or dynamic loads developed on prosthetic joints during their use. Resulting advantages include, but are not limited to: longer functional prosthetic life; reduced frequency of surgical procedures for repair or replacement of prosthetics; reduced rate of prosthetic-associated complications such as osteolysis and/or joint dislocation; and enhanced economic benefits proceeding from these advantages.

### CROSS-REFERENCE TO RELATED APPLICATION

This application claims the priority date of Provisional Patent Application No. 61666894, “Improved Prosthetic Joints”, filed on Jul. 1, 2012 by John Michael Pinneo.

### REFERENCES CITED

### Other Publications

Mallinson, J. C., IEEE Transactions on Magnetics, Vol. MAG-9, No. 4, December 1973,678-682.

Dai, et al., Chinese Medical Journal 2010; 123(23): 3451-3454

### BACKGROUND OF THE INVENTION

This invention pertains to the field of prosthetic joints employed in treatment of patients using procedures generally known as arthroplasty.

Prosthetic joints are implanted medical devices that emulate the functionality of natural joints and restore function to patients whose natural joint function has been compromised. In the United States, millions of people have artificial hips, knees, and other implanted prosthetic joints. Many projections estimate an annual need for 500,000 total hip replacements and 3,000,000 total knee replacements in the United states by 2030, at a cost exceeding $40 billion. The increase in total hip and knee replacements is a world-wide trend, and health care costs for joint replacements are rising rapidly in all developed nations.

Prosthetic joints present a range of difficult design and bioengineering problems. Their operating environment is chemically corrosive and biologically active. They must form robust, reliable interfaces with living tissue, while handling a complex variety of static and dynamic loads resulting from their users' activities. Finally, they must function reliably for decades in increasingly younger and more active patients.

To date, the engineering problems associated with prolonged service life of prosthetic implants have been imperfectly solved. Prosthetic joints fail in several modes, including mechanical wear at their opposed load-bearing surfaces, outright fracture of implanted components, and loosening at the interface between implanted prosthetic components and the host tissue, usually bone. Debris from wear processes can induce degradative biological processes that lead to implant failure and associated local osteolysis as well as causing adverse systemic reactions in implant patients.

In addition to limited service life, some prosthetic joints exhibit undesirable limitations even when properly functioning. An example is the tendency of hip prostheses to dislocate under certain conditions of load and positioning. It is common that recipients of prosthetic hip joints, whether of a total hip replacement or of a resurfacing procedure in which the femoral head and acetabulum are “resurfaced” with prosthetic components, are cautioned not to rotate the implanted joint beyond certain angular limits in order to avoid disarticulation, or dislocation of the joint. Such limitations are not exclusive to hip joint prosthetics.

Many approaches have been taken in the prior art to improve service life of prosthetic joints and to mitigate their many associated limitations and drawbacks. The utility of prosthetic joints has strongly motivated research and development of the art, resulting in thousands of published research reports and numerous patents on improvements in prosthetic joints. However, despite the extensive prior art in this field, it is accurate to note that prosthetic joints that can perform satisfactorily for the lifetime of their users remain a goal for the future, rather than a current reality.

Two major areas of research have focused on new materials and improved structures for prosthetic joints.

A wide variety of materials have been explored for use in prosthetic joints. Those skilled in this field of art will be familiar with UHMWPE (ultra high molecular weight polyethylene) and other plastics, metals including CoCrMo alloys, titanium alloys, various ceramics such as alumina and silicon nitride, a range of coating materials including ceramics and diamond or diamond-like carbon, and combinations of the foregoing. Much work has been done to clarify the role of different materials in producing wear debris that triggers adverse local and systemic reactions through various complex biochemical reactions.

As well, a wide range of mechanical engineering solutions have been devised in the continued quest for improved prosthetic joint performance. Spongiform titanium prosthetics have been developed in an effort to provide a better match to the host bone's elastic modulus. In hip prosthetics, sliding shells have been interposed between the femoral head and the acetabular cup to reduce wear and torque loads resulting from contact between the edge of the acetabular cup and portions of the femoral head at high joint angle excursions. Various mechanical means of improving the interface between the implant and the host bone have been attempted, including threaded interfaces, improved bone cement formulations, and surface coatings to promote post-implantation osteointegrative processes.

In all of the prior art, mechanical load carried by prosthetic joints is acknowledged as a universal factor in determining the useful life of joints. Despite the complex nature of joint wear phenomena, it is the case that simply reducing mechanical load on the joint reduces its wear rate.

Much work has been done to elucidate the mechanisms by which static and dynamic mechanical loads appear, or are resolved within, prosthetic joints. Advanced structural mechanics modeling methods, coupled with in vitro and in vivo load measurements have revealed much about how implant topology, surface finish, and the local bioenvironment affect the distribution of mechanical forces that are imposed on implanted prosthetic joints.

Mechanical loads create forces that degrade prosthetic joints by causing wear at opposed moving surfaces, by causing outright fracture or breakage of joint components, and by degrading the interface between the implant and surrounding host tissues. These forces are both static and dynamic. Thus, a patient with a prosthetic hip implant may exert a 100 pound static force on the joint when standing at rest. This force may be briefly multiplied several fold as the patient walks, runs, or otherwise engages in ordinary activities. These peak forces may fracture joint components or damage joint/tissue interfaces.

One attempt to mitigate load-driven prosthetic wear through use of magnets is known in the prior art. In Dai, et al. (see References Cited), researchers installed magnets into models of human hip joints. A single magnet was installed in a model of an acetabular cup and a single magnet was installed in a model of a femoral head. The single magnets in each component were arranged so that they generated a repulsive force when brought into proximity as would be the case in an implanted hip prosthesis. Wear measurements were performed, and a reduction in wear of joint model components was noted.

Unfortunately, at least two aspects of the work by Dai, et al., prevented its progression to clinical utility. One unfavorable aspect involved the use of single magnets, one each disposed in the opposing model joint components. This configuration is known by those skilled in the art of applying permanent magnets to the generation of controllable force to be inefficient to a disabling extent in this application. In particular, it is known that a single pair of magnets in opposition exhibit a magnetic field configuration that puts fully half of the available magnetic flux out of the zone of interaction between the two opposed magnets, thereby greatly reducing the force that may be developed between the opposed magnets, as shown in FIG. 1. This was a critical impediment of the work by Dai, et al., in that the space available for disposing magnets within prosthetic joint components is extremely limited, and inefficient use of magnetic flux precludes or greatly reduces utility by limiting the magnitude of wear-mitigating force that may be generated.

A second impediment to utility in the work by Dai, et al., was noted by the authors who stated that they were required to employ shielding to restrict the presence of magnetic flux from regions other than the desired region of opposition between the two magnets. Materials that are effective magnetic shields are comprised principally of ferromagnetic materials such as iron, nickel, and the like. Many of these materials are not compatible with long-term use when implanted in the human body, and therefore present hazards that must be overcome with the use of biocompatible coatings, thereby introducing additional possibilities for implant failure. As well, the necessity for magnetic shielding in the Dai, et al., work further reduces the space available for disposition of magnets, thereby reducing the magnetic force and wear reduction that may be achieved. Thus, the work of Dai, et al., did not disclose or suggest a means of improving the clinical utility or performance of prosthetic joints.

Wear phenomena therefore continue to severely limit the utility of prosthetic joints as a beneficial medical technology.

### DESCRIPTION OF FIGURES

FIG. 1 depicts the pattern of the extended magnetic field (2) that is generated by two bipolar magnets placed so as to generate a repulsive force between the two magnets. Each magnet (1) exhibits a south magnetic pole (3) and a north magnetic pole (4). Arrows are placed to indicate the direction of magnetic flux within each magnet according to the conventions of the magnetic arts in which an arrowhead depicts the direction of the north magnetic pole.

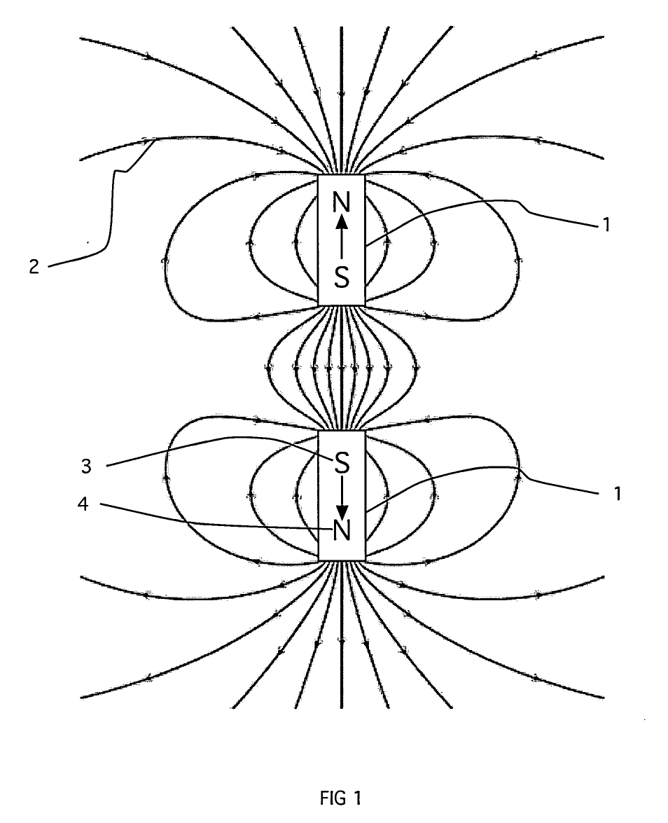

FIG. 2 depicts a schematic side sectional view of several bipolar permanent magnets (1) configured in a linear Halbach array, a well known example of magnetic arrays that concentrate or intensify magnetic fields in a directional manner. Schematic magnetic field lines (2) illustrate the Halbach array's concentration of magnetic flux on one side of the array and the simultaneous diminution of magnetic flux on the opposite side of the array. Magnetic south poles (3) and magnetic north poles (4) are shown to illustrate the disposition of pole orientations in the Halbach configuration.

FIG. 3 shows a schematic side sectional view of two Halbach magnet arrays interacting so as to develop a mutually repulsive force at their regions of maximum magnetic field intensity. Each magnet (1) has a south (3) and a north (4) pole arranged as shown, with schematic magnetic field lines (2) showing the intensity and extent of the arrays' magnetic fields.

FIG. 4 depicts a schematic sectional view of two curved arrays of magnets (1)disposed in a mutually repulsive Halbach configuration. Each array is shown as positioned behind opposed curvilinear surfaces that are free to slide with respect to one another (5). Surfaces (5) are representative of the opposed surfaces of the acetabular cup and the femoral head of a prosthetic hip joint, as well as other types of prosthetic joints that are operable with this invention. Explicit labels for north and south magnetic poles has been omitted for clarity, while depicted arrows indicate the direction of magnetic flux according to the art.

FIG. 5 depicts a schematic view of two arrays of magnets (1), one array being disposed within an acetabular cup component (6) and the other array being disposed within a femoral head (7) component of a prosthetic hip joint that includes a femoral neck (8). The magnet arrays are configured to produce a mutually repulsive force as shown by the arrows that depict the direction of magnetic flux. Each magnet array is disposed behind the sliding surfaces (5) of the components (6 and 7) within which they are secured.

### BRIEF SUMMARY OF THE INVENTION

Arrays consisting of pluralities of magnets are employed to redistribute forces within prosthetic joints, with resulting benefits to prosthetic joint performance and longevity. The invention overcomes prior barriers that have to date prevented the use of magnets to mitigate wear and failure processes in prosthetic joints.

### DETAILED DESCRIPTION OF INVENTION

This invention is directed towards redistributing static and dynamic mechanical forces resolved on prosthetic joints by novel means.

Description of the invention: In a prosthetic joint, multiple permanent magnets are disposed so as to redistribute mechanical loads imposed on the joint with the result that a portion of the applied load that would normally appear, or be resolved at, the joint's opposed moving surfaces is carried by the magnets, thereby reducing the force carried by the interface.

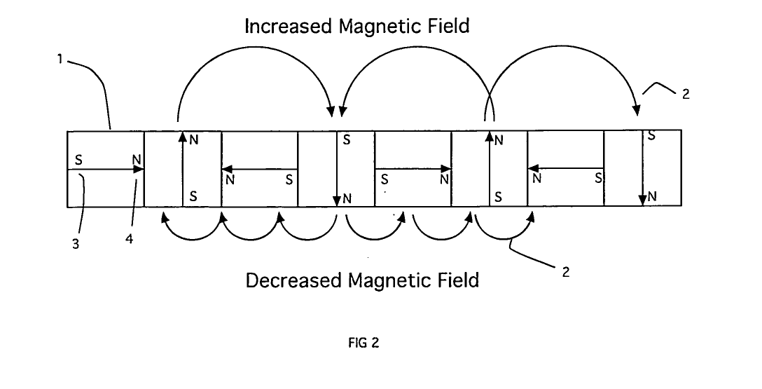

Referring to FIG. 2, a linear array of permanent magnets (1) is depicted in one configuration (the Halbach configuration) that is known to concentrate magnetic flux on one side of the array while diminishing magnetic flux on the opposite side, as shown by the magnetic field line (3) illustration. South (3) and north (4) magnetic poles are arranged as shown to achieve the flux concentration, or flux directive properties exhibited by Halbach arrays. To the extent that external magnetic fields from magnets not part of this array exert forces on the array, those forces will be enhanced or diminished according to whether external magnetic fields interact with the array principally in the region having enhanced magnetic flux or in the region having diminished magnetic flux. Magnets (1) are depicted as being rectilinear, but may in fact be comprised of any shape(s) consistent with the requirements of a specific application. In particular, magnet arrays with flux concentrating properties are known in which individual magnets have other than rectilinear shapes, including trapezoids, arcs, cylinders, and spheres.

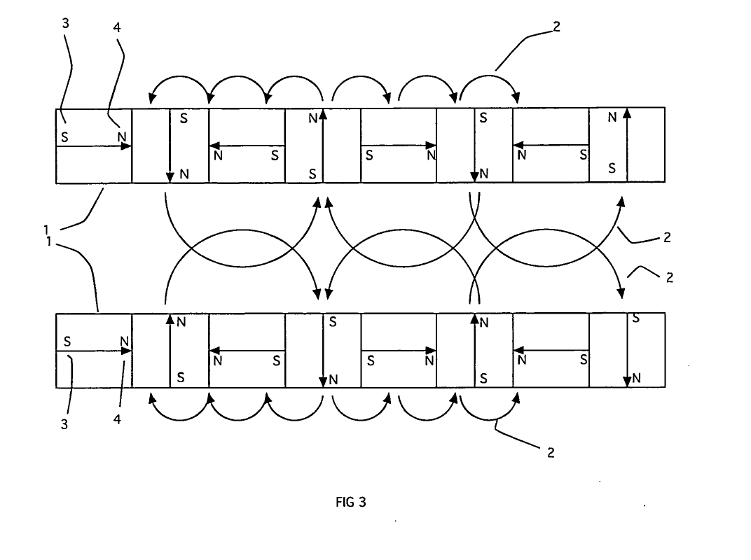

Referring to FIG. 3, two linear arrays of magnets (1) are shown in with south (3) and north (4) magnetic poles configured such that their magnetic fields (2) interact most strongly near the sides of the arrays at which magnetic flux is concentrated, and the mutually repulsive magnetic force that develops between the two arrays is thereby maximized.

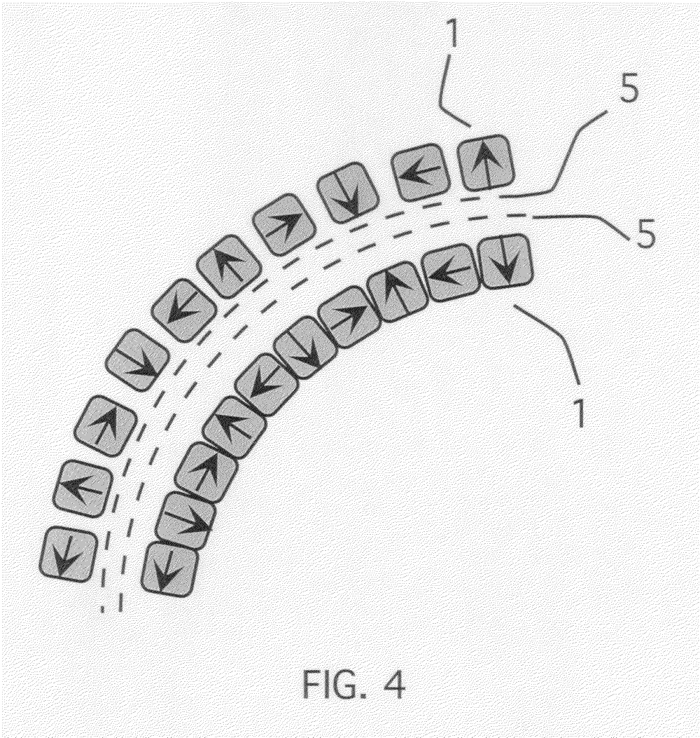

Referring to FIG. 4, two curvilinear arrays of magnets (1) are depicted schematically as being disposed behind two curvilinear surfaces (5) that are in close opposition or in contact with each other. The magnet arrays are configured as shown by arrows whose labeling has been omitted for clarity in a manner to cause mutual repulsion between the two magnet arrays, this repulsive force being transmitted between the two arrays across the sliding interface formed by the two surfaces (5).

Mechanical loads applied to a prosthetic joint may be considered to resolve into various forces that can be described by vectors, that is, having magnitudes and directions. In this description, we designate any force that tends to reduce the distance between the opposed moving joint components as being compressive. Compressive forces bring joint interface surfaces into physical contact, thereby causing mechanical wear of the joint surfaces. Reduction of these forces by directing all or a portion of them away from the joint interface will decrease joint interface wear. Reduction of these forces may be achieved by superimposing forces generated by arrays of magnets configured to counteract compressive mechanical forces at the joint interface.

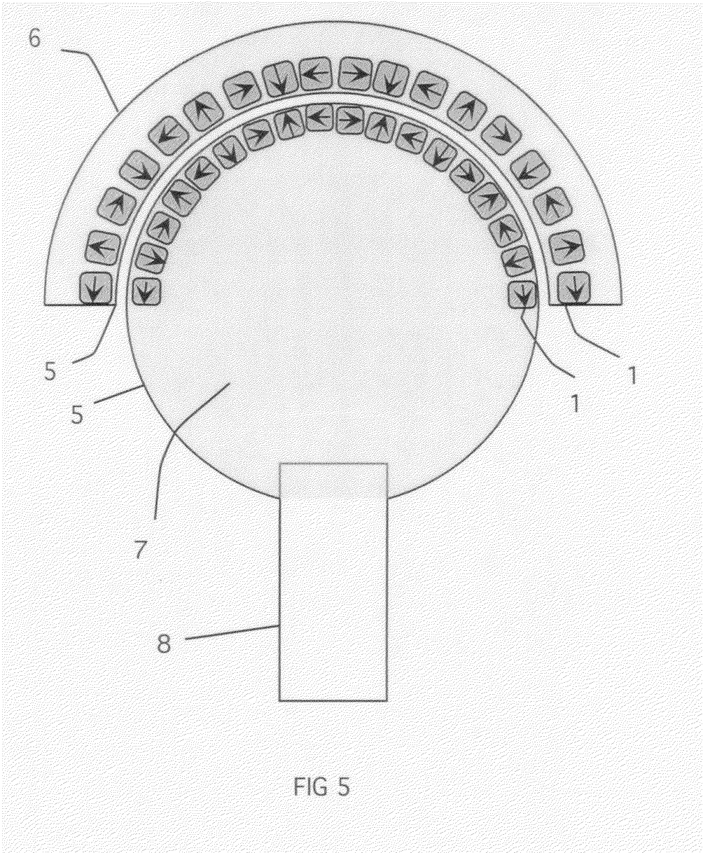

Referring now to FIG. 5, and using a prosthetic human hip joint as a non-limiting example, at least two magnets (an array) are disposed within the acetabular cup and at least two magnets (an array) are disposed within the femoral head with their magnetic polarities arranged such that a repulsive force arises between the acetabular cup and femoral head when they are positioned as they would be in an implanted joint.

In this configuration, a portion of the compressive mechanical force that is caused by load on the joint and that appears at the joint interface is reduced by repulsive force generated by the magnet arrays disposed within the opposed joint components. The reduced force appears as repulsive force exerted on the magnets and is initially resolved at the interface between the magnets and their surrounding implant components. Thus, while the overall force resolved by the joint structure still remains equal to the load applied to the joint, that portion of the force responsible for interface wear is reduced, thus reducing joint interface wear.

It will be apparent to those skilled in the art that the relationship between magnet repulsive force and joint position (where joint position includes the relative positions of joint components in three axes of translation and rotation) may be advantageously tailored by the particulars of magnet disposition within the joint components. It is possible thereby to, as a non-limiting example, cause the magnetic repulsive force to reverse sign such that it tends to hold the joint components in proximity to one another if the joint is rotated to a non-operable position, thereby reducing or preventing dislocation of the joint.

It will be apparent to those skilled in the art that peak forces generated on the joint by dynamic mechanical loads may be reduced by mounting all or some of the magnets disposed within at least one of the joint components with compliant material, the magnets' increased range of motion acting to lengthen the duration of force peaks while reducing their magnitude.

In one embodiment of this invention, at least one of the magnet arrays disposed within the joint components comprises a Halbach array or other array of magnets that concentrates magnetic flux in one region of space while diminishing magnetic flux in another region of space. A Halbach array is a configuration of magnetic pole orientations known to the art to effectively concentrate magnetic flux generated by an array of magnets so that increased magnetic interaction forces may be generated using a smaller volume of magnetic material than would be possible without a Halbach configuration. Furthermore, by directionally concentrating its magnetic field, a Halbach array minimizes the spatial extent of the magnetic field, thereby reducing the need for magnetic shielding to exclude the magnetic field from regions where its presence would be disadvantageous.

In another embodiment of this invention, the disposition of at least one of the magnet arrays within the joint components departs from a substantially straight line, thereby approximating at least one curved line.

In another embodiment of this invention, the disposition of at least one of the magnet arrays within the joint components departs from a substantially planar surface, thereby approximating at least one curved surface.

In another embodiment of this invention, at least one of the magnets within at least one of the magnet arrays disposed within the joint components is fixed in position by means that afford a specified degree of movement of the magnet in response to a force applied to the magnet.

In another embodiment of this invention, magnets comprising magnetic arrays that depart from substantially straight lines or substantially planar surfaces are shaped so as to minimize empty volumes between adjacent magnets, thereby maximizing the amount of magnetic material disposed within a given region.

In another embodiment of this invention, at least some of the magnets disposed within a prosthetic joint are at least in part in contact with the local biological environment.

In another embodiment of this invention, a prosthetic joint is constructed with a plurality of magnets disposed within one component of the prosthetic joint, while the opposed component of the prosthetic joint may contain or embody a region or regions that consist of a metal or metal alloys that may exhibit ferromagnetic properties.

In another embodiment of this invention, a prosthetic joint is constructed such that magnetic properties of at least one of the magnets that comprise at least one plurality of magnets disposed within at least one component of the prosthetic joint may be altered so as to alter the function of the joint.

In another embodiment of this invention, a prosthetic joint is constructed such that at least a plurality of magnets is disposed so as to reduce the probability joint dislocation.

In another embodiment of this invention, a prosthetic joint incorporating at least one plurality of magnets is constructed so as to facilitate surgical procedures associated with implantation and or explantation and or repair of the implanted joint, and or facilitate patient recovery from said procedures.

While embodiments and applications of this invention have been shown and described, it would be apparent to those skilled in the art that many more modifications than mentioned above are possible without departing from the inventive concepts therein.

I claim:

1. A prosthetic joint, at least one component of which has within it a plurality of magnets disposed so as to provide increased utility of the prosthetic joint.

2. A prosthetic joint as in claim 1, in which within at least two components of the prosthetic joint are disposed magnets such that each component contains a plurality of magnets.

3. A prosthetic joint as in claim 1, in which at least one plurality of magnets exhibits flux concentrating or flux directive properties.

4. A prosthetic joint as in claim 1, in which at least one plurality of magnets is configured so as to inhibit at least in part motion of the joint into inoperable positions.

5. A prosthetic joint as in claim 1, in which at least one plurality of magnets is configured to provide at least one specified correspondence between relative positions of the joint's components and magnetic forces developed at different joint component relative positions.

6. A prosthetic joint, at least two components of which have within each component a plurality of magnets disposed so as to modify the magnitude and or the distribution and or the temporal characteristics of forces within the joint and or at its interfaces and or its interfaces with its surroundings, said forces arising through operation of the joint.

7. A prosthetic joint as in claim 6, in which at least one plurality of magnets exhibits flux concentrating or flux directive properties.

8. A prosthetic joint as in claim 6, in which at least one plurality of magnets is configured so as to inhibit at least in part motion of the joint into inoperable positions.

9. A prosthetic joint as in claim 6, in which at least one plurality of magnets is configured to provide at least one specified correspondence between relative positions of the joint's components and magnetic forces developed at different joint component relative positions.

10. A prosthetic joint, in which at least a plurality of magnet arrays each consisting of a plurality of magnets disposed so as to reduce and or slow wear processes exhibited by said prosthetic joint, said processes covarying with mechanical loads imposed on the prosthetic joint during its operation.

11. A prosthetic joint as in claim 10, in which at least one plurality of magnets exhibits flux concentrating or flux directive properties.

12. A prosthetic joint as in claim 10, in which at least one plurality of magnets is configured so as to inhibit at least in part motion of the joint into inoperable positions.

13. A prosthetic joint as in claim 10, in which at least one plurality of magnets is configured to provide at least one specified correspondence between relative positions of the joint's components and magnetic forces developed at different joint component relative positions.

14. A prosthetic joint as in claim 10, in which at least one plurality of magnets is configured such that the probability of joint dislocation is reduced.
